(19) United States
(12) Patent Application Publication
(10) Pub. No.: US 2025/0222036 A1
(43) Pub. Date: Jul. 10, 2025

(54) DELIVERY OF DISSOCIATED ISLETS CELLS WITHIN MICROPOROUS ANNEALED PARTICLE SCAFFOLD TO TREAT TYPE 1 DIABETES

(71) Applicant: University of Virginia Patent Foundation, Charlottesville, VA (US)

(72) Inventors: Donald Richieri Griffin, Charlottesville, VA (US); Colleen Roosa, Charlottesville, VA (US); Mingyang Ma, Charlottesville, VA (US); Preeti Chhabra, Waynesboro, VA (US); Kenneth Brayman, Charlottesville, VA (US)

(73) Assignee: University of Virginia Patent Foundation, Charlottesville, VA (US)

(21) Appl. No.: 18/850,952

(22) PCT Filed: Mar. 27, 2023
(86) PCT No.: PCT/US2023/065006

§ 371 Date: Sep. 25, 2024

Related U.S. Application Data
(60) Provisional application No. 63/323,650, filed on Mar. 25, 2022.

(51) Int. Cl. A61K 35/39; A61K 9/16; A61K 38/28; A61P 3/10

(52) U.S. Cl. CPC A61K 35/39; A61K 9/1641; A61K 38/28; A61P 3/10

(57) ABSTRACT

Provided are compositions that include single cell suspensions of pancreatic islet cells, pancreatic islet-like cells derived from iPS cells, or combinations thereof, wherein the cells are present within a MAP scaffold and/or are encapsulated by MAPs. In some embodiments, the MAP scaffold and/the MAPs have a polymer backbone that includes poly(ethyleneglycol) (PEG), hyaluronic acid, polyacrylamide, polymethacrylate, alginate, collagen, or any combination thereof. Also provided are methods for using the presently disclosed compositions for treating Type 1 diabetes, for example by administering to a subject with Type 1 diabetes such a composition via a route and in an amount effective for treating the Type 1 diabetes in the subject. In some embodiments, the administering includes injecting the composition into a kidney capsule, subcutaneously, intraperitoneally, into adipose tissue, intramuscularly, intrahepatically, and/or intrapancreatically into the subject.

### CROSS REFERENCE TO RELATED APPLICATIONS

The presently disclosed subject matter claims priority to and the benefit of U. S. Provisional Patent Application Ser. No. 63/32,650, filed Mar. 25, 2022, the disclosure of which is herein incorporated by reference in its entirety.

### GOVERNMENT INTEREST

This invention was made with government support under Grant No. R21EB028971 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 2,723 byte UTF-8-encoded XML file created on Mar. 23, 2023 and entitled “3062_179_PCT.xml”. The Sequence Listing submitted via Patent Center is incorporated herein by reference in its entirety.

### TECHNICAL FIELD

The presently disclosed subject matter relates generally to compositions and methods for treating diseases, disorders, and conditions in subjects in need thereof. In some embodiments, the subject has Type 1 diabetes and the compositions and methods of the presently disclosed subject matter are used for treating the Type 1 diabetes by introducing into the subject an effective amount of a composition comprising a microporous annealed particle (MAP) scaffold and a single cell suspension present therein, wherein the single cell suspension comprises pancreatic islet cells, pancreatic islet-like cells derived from induced pluripotent stem (iPS) cells, or a combination thereof.

### BACKGROUND

Type 1 diabetes (TID) affects 1.6 million Americans (CDC, 2020), and is most frequently caused by the autoimmune destruction of the insulin-producing beta cells in the pancreas which can lead to serious or fatal health complications if left untreated. Most patients with TID manage their diabetes through a combination of blood glucose monitoring (e.g., finger prick testing) and systemic exogenous insulin delivery (e.g., intramuscular injection). While advanced monitoring and delivery techniques do exist (e.g., implantable insulin pumps), the only clinical approach to patient management of blood glucose that does not require exogenous insulin is whole pancreas transplantation or pancreatic islet delivery via portal vein injection. Unfortunately, donor islets are rare; only 1,086 patients were treated with islet transplantation worldwide between 1999 and 2015 (Collaborative Islet Transplant Registry, 2015). This scarcity of islets has prompted the search for alternative sources of beta cells.

Stem cell-based approaches using induced pluripotent stem cells (iPSCs) and pancreas-derived multipotent precursor cells are currently being explored for beta cell production, and represent a promising cell source for treatment of TID (Chhabra & Brayman, 2018). Outside of stem-cell derived beta cells, native beta cells derived from dissociated islets are a relatively accessible cell source (i.e., do not require extensive differentiation protocols) that can serve as a model cell type for stem-cell derived approaches. However, several studies have shown that both dissociated islets and stem-cell derived beta cells typically require cell aggregation in vitro prior to implantation (Tsang et al., 2007; Kodama et al., 2009; Hilderink et al., 2015; Yu et al., 2018; Lebreton et al., 2019; Tran et al., 2020; Nakayama-Iwatsuki et al., 2021. This facilitates biochemical and biomechanical cell interactions that are necessary for cell differentiation, survival, and function. Additionally, it has been shown that dissociated islets cannot survive in vivo without close cellular communication (Wang & Rosenberg, 1999; Benninger & Piston, 2014; Li, 2020) and extracellular matrix mimicking ligands (Wang & Rosenberg, 1999; Suarez-Pinzon et al., 2005; Lin & Anseth, 2009; Lin et al., 2011) The formation of “pseudo-islets” typically involves enzymatically dissociating whole islets into single cells or differentiating beta cell precursors, then forming islet-like clusters of a desired size that promote better nutrient diffusion and cell survival. These pseudo-islet clusters can be formed in microwells (O'Sullivan et al., 2010), Matrigel (O'Berg-Welsh, 2001), or via hanging drop method (Gao et al., 2016). Another alternative delivery method is via synthetic microporous scaffolds (Lin et al., 2011; Gao et al., 2011; Gao et al., 2016; Youngblood et al., 2019; Tran et al., 2020), which allows for cell self-organization within pores that can mimic native islet structure and facilitate cell-cell signaling. However, cell clustering techniques typically require a 3-5 day “pre-conditioning” period in vitro prior to implantation.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to compositions comprising, consisting essentially of, or consisting of a microporous annealed particle (MAP) scaffold and a single cell suspension present therein. In some embodiments, the single cell suspension comprises, consists essentially of, or consists of one or more cells that are capable of releasing insulin in response to an elevated glucose concentration encapsulated by microporous annealed particles (MAPs). In some embodiments, the MAP scaffold comprises a polymer backbone comprising, consisting essentially of, or consisting of poly(ethyleneglycol) (PEG), hyaluronic acid, polyacrylamide, polymethacrylate, alginate, collagen, or any combination thereof. In some embodiments, the MAP scaffold comprises one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer, optionally wherein the annealing monomer is a MethMal annealing macromer. In some embodiments, the MAP scaffold has a porosity of about 10 μm to about 200 μm. In some embodiments, the MMP-2 degradable crosslinker comprises, consists essentially of, or consists of an amino acid sequence comprising, consisting essentially of, or consisting of Ac-GCGPQGIAGQDGCG-NH₂ (SEQ ID NO: 1). In some embodiments, the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof. In some embodiments, the one or more cells that are capable of releasing insulin in response to an elevated glucose concentration are human cells or are derived from human cells.

The presently disclosed subject matter also relates in some embodiments to compositions comprising, consisting essentially of, or consisting of single cell suspensions comprising, consisting essentially of, or consisting one or more cells that are capable of releasing insulin in response to an elevated glucose concentration encapsulated by microporous annealed particles (MAPs). In some embodiments, the MAPs comprise a polymer backbone comprising, consisting essentially of, or consisting of poly(ethyleneglycol) (PEG), hyaluronic acid, polyacrylamide, polymethacrylate, alginate, collagen, or any combination thereof. In some embodiments, the MAPs comprise one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH2 (SEQ ID NO: 2); and an annealing monomer, optionally wherein the annealing monomer is a MethMal annealing macromer. In some embodiments, the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof. In some embodiments, the MMP-2 degradable crosslinker comprises, consists essentially of, or consists of an amino acid sequence comprising, consisting essentially of, or consisting of Ac-GCGPQGIAGQDGCG-NH2 (SEQ ID NO: 1). In some embodiments, the one or more cells that are capable of releasing insulin in response to an elevated glucose concentration are human cells or are derived from human cells.

The presently disclosed subject matter also relates in some embodiments to methods for treating diabetes. In some embodiments, the methods comprise administering to a subject with diabetes one or more of the compositions disclosed herein via a route and in an amount effective for treating the diabetes in the subject. In some embodiments, the diabetes is Type 1 diabetes. In some embodiments, the diabetes is Type 2 diabetes. In some embodiments, the administering comprises injecting the composition into a kidney capsule, subcutaneously, intraperitoneally, into adipose tissue, intramuscularly, intrahepatically, and/or intrapancreatically into the subject. In some embodiments, the composition comprises one or more cells that are capable of releasing insulin in response to an elevated glucose concentration that are human cells or are derived from human cells.

The presently disclosed subject matter also relates in some embodiments to use of compositions comprising, consisting essentially of, or consisting of a microporous annealed particle (MAP) scaffold and a single cell suspension present therein, wherein the single cell suspension comprises one or more cells that are capable of releasing insulin in response to an elevated glucose concentration, for treating diabetes, wherein the composition is formulated for administration to a subject in need thereof via a route and in an amount effective for treating the diabetes in the subject. In some embodiments, the MAP scaffold comprises a polymer backbone comprising, consisting essentially of, or consisting of poly(ethyleneglycol) (PEG), hyaluronic acid, polyacrylamide, polymethacrylate, alginate, collagen, or any combination thereof. In some embodiments, the MAP scaffold comprises one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH2 (SEQ ID NO: 2); and an annealing monomer, optionally wherein the annealing monomer is a MethMal annealing macromer. In some embodiments, the MAP scaffold has a porosity of about 10 μm to about 200 μm. In some embodiments, the MMP-2 degradable crosslinker comprises, consists essentially of, or consists of an amino acid sequence comprising, consisting essentially of, or consisting of Ac-GCGPQGIAGQDGCG-NH2 (SEQ ID NO: 1). In some embodiments, the composition is administered administering by injecting the composition into a kidney capsule, subcutaneously, intraperitoneally, into adipose tissue, intramuscularly, intrahepatically, and/or intrapancreatically into the subject. In some embodiments, the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof. In some embodiments, the one or more cells that are capable of releasing insulin in response to an elevated glucose concentration are human cells or are derived from human cells. In some embodiments, the diabetes is Type 1 diabetes. In some embodiments, the diabetes is Type 2 diabetes.

The presently disclosed subject matter also relates in some embodiments to compositions for use in treating diabetes in a subject in need thereof, the composition comprising, consisting essentially of, or consisting of a single cell suspension comprising, consisting essentially of, or consisting of one or more cells that are capable of releasing insulin in response to an elevated glucose concentration encapsulated by microporous annealed particles (MAPs). In some embodiments, the MAPs comprise a polymer backbone comprising, consisting essentially of, or consisting of poly(ethyleneglycol) (PEG), hyaluronic acid, polyacrylamide, polymethacrylate, alginate, collagen, or any combination thereof. In some embodiments, the MAPs comprise one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH2 (SEQ ID NO: 2); and an annealing monomer, optionally wherein the annealing monomer is a MethMal annealing macromer. In some embodiments, the MMP-2 degradable crosslinker comprises, consists essentially of, or consists of an amino acid sequence comprising, consisting essentially of, or consisting of Ac-GCGPQGIAGQDGCG-NH2 (SEQ ID NO: 1). In some embodiments, the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof. In some embodiments, the diabetes is Type 1 diabetes. In some embodiments, the diabetes is Type 2 diabetes.

Accordingly, it is an object of the presently disclosed subject matter to provide compositions and methods for treating diseases, disorders, and conditions in subjects in need thereof. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTION OF THE FIGURES

FIG. 1A: Overview of whole islet dissociation, filtration, and implantation with MAP scaffold under the renal subcapsular space. The MAP scaffold contains heparin-μislands, which has previously demonstrated to significantly improve vascularization within the scaffold (see Pruett et al., 2021a).

FIG. 1B: 3D rendering of MAP scaffold pores (green dextran) and heparin-μisland particles (red) in Imaris software (Oxford Instruments).

FIGS. 2A-2E. Results from viability and GSIS assays on dissociated islet cells incubated with and without MAP gel in a transwell plate. FIG. 2A. Percentage of live cells imaged at 1, 24, 48, and 72 hr. p<0.05, two-way ANOVA with Sidak's multiple comparison test (GraphPad Prism). FIG. 2B. Percent viability of dissociated cells from 50, 12.5, or 2.5 islets incubated in MAP gel for 72 hours. p<0.05 one-way ANOVA with Tukey's multiple comparison test (GraphPad Prism). FIG. 2C. Insulin concentration detected by ELISA in the high and low glucose buffers that dissociated islets cells were exposed to for 1 hour each after 1, 24, 48, and 72 hr incubation with or without MAP scaffold (GSIS assay). p<0.05, two-way ANOVA with Sidak's multiple comparison test (GraphPad Prism); FIGS. 2D and 2E. Representative live/dead images of dissociated islets cells incubated with or without MAP scaffold at 1-hour (FIG. 2D) and 72-hour (FIG. 2E) timepoints. Live cells were stained with fluorescein diacetate (FDA) and dead cells stained with propidium iodine (PI) and imaged at 4× (scale bar=500 μm).

FIGS. 3A-3C: Blood glucose data and representative immunohistochemistry (IHC) images from syngeneic implant. FIG. 3A. Daily blood glucose levels (mg/dL) of recipient diabetic mice (n=6 per condition). Left nephrectomy at day 40 was done to confirm return of hyperglycemia. FIG. 3B. Representative IHC image of implant site that received dissociated islets only. FIG. 3C. Representative IHC image of MAP implant site that contained dissociated islet cells. Dark-brown DAB chromogen stain indicates positive staining for insulin (indicated by black arrows). Scale bar=150 μm.

FIG. 4. Photomicrograph of an exemplary MAP microgel with encapsulated cells. The MAPs are shown in purple and the cells are shown in green.

FIGS. 5A-5G. Cytokine content of gel implants with beta cells. Cytokine panel from a murine multiplex proinflammatory cytokine assay (Luminex) performed on biomaterial explants 7-days post implantation. At the time of implantation, both implant groups contained cultured Beta TC-6 cells (ATCC, Inc.) at a concentration of 1×10⁶ cells per μL. The two experimental groups were microporous annealed particle scaffold (MAP) and a chemically identical nanoporous hydrogel control (NP). Cytokines assayed were interferon gamma (IFNγ; FIG. 5A), interleukin-6 (IL-6; FIG. 5B), interleukin-1 alpha (IL-1α; FIG. 5C), interleukin-1 beta (IL-1B; FIG. 5D), interleukin-10 (IL-10; FIG. 5E), interleukin-17 (IL-17; FIG. 5F), and tumor necrosis factor alpha (TNFα; FIG. 5G). N=5 per group; * p<0.05.

### DETAILED DESCRIPTION

Type 1 diabetes (TID) is caused by the autoimmune loss of insulin-producing beta cells in the pancreas. The only clinical approach to patient management of blood glucose that does not require exogenous insulin is pancreas or islet transplantation. Unfortunately, donor islets are scarce and there is substantial islet loss immediately after transplantation due, in part, to the local inflammatory response. The delivery of stem cell-derived beta cells (e.g., from induced pluripotent stem cells) and dissociated islet cells hold promise as a treatment for TID; however, these cells typically require re-aggregation in vitro prior to implantation.

Microporous scaffolds have shown potential to serve as a vehicle for organization, survival, and function of insulin-producing cells. As disclosed herein, the use of microporous annealed particle (MAP) scaffold for delivery of enzymatically dissociated islet cells, a model beta cell source, within the scaffold's interconnected pores is described. It was found that MAP-based cell delivery enables survival and function of dissociated islets cells both in vitro and in an in vivo mouse model of TID.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments±10%, in some embodiments±5%, in some embodiments ±1%, in some embodiments±0.5%, and in some embodiments±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 1:

TABLE 1
Amino Acid Codes and Functionally Equivalent Codons

| | 3-Letter | 1-Letter | Functionally Equivalent |
|---|---|---|---|
| Full Name | Code | Code | Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is me\ant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the compositions of the presently disclosed subject matter, 10 and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the compositions of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

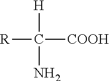

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in Table 2:

TABLE 2
Exemplary Conservative Amino Acid Substitutions

| Group | Characteristics | Amino Acids |
|---|---|---|
| A. | Small aliphatic, nonpolar or slightly | Ala, Ser, Thr, Pro, Gly |
| | polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes, or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged methylene phosphonate, phosphoramidate, bridged phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, the phrase “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses;

domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Compositions and Pharmaceutical Compositions

### II.A. Compositions Generally

In some embodiments, the presently disclosed subject matter relates to compositions comprising, consisting essentially of, or consisting of a microporous annealed particle (MAP) scaffold and a single cell suspension present therein, wherein the single cell suspension comprises, consists essentially of, or consists of one or more cells that are capable of releasing insulin in response to an elevated glucose concentration. In some embodiments, the MAP scaffold comprises a polymer backbone comprising, consisting essentially of, or consisting of poly(ethyleneglycol) (PEG), hyaluronic acid, polyacrylamide, polymethacrylate, alginate, collagen, or any combination thereof. In some embodiments, the MAP scaffold comprises one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer. Exemplary MAP scaffolds and methods for producing the same are disclosed in U.S. Patent Application Publication No. 2021/0052779 and PCT International Patent Application Publication No. WO 2021/113812, both of which are incorporated herein by reference in their entireties.

The MAP scaffolds are designed to deliver cells that are capable of releasing insulin in response to an elevated glucose concentration in a subject in need thereof. Exemplary cells that are capable of releasing insulin in response to an elevated glucose concentration include endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof. Endocrine precursor cells and methods for isolating, preparing, and using them are disclosed, for example, in U.S. Patent Application Publication No. 2012/0039955 and U.S. Pat. Nos. 8,859,286 and 9,650,610, each of which is incorporated by reference herein.

In some embodiments, the annealing monomer is a MethMal annealing macromer. As used herein, the term “MethMal” refers to a heterofunctional maleimide/methacrylamide 4-arm PEG macromer as described in Pfaff et al., 2021. As disclosed therein, MethMal annealing macromers can be employed for photopolymerization with various polymer backbones, including but not limited to the polymer backbones disclosed herein (e.g., PEG-Maleimide). Methods for synthesizing PEG macromers are known and include the exemplary method disclosed in Pfaff et al., 2021. That method included a two-step, one pot modification of 4-arm 20 kDa PEG-maleimide using 2-aminoethanethiol followed by amidation using methacrylic acid via DMTMM (4-(4,6-dimethoxy-1,3,5-triazin-2-yl)-4-methyl-morpholinium chloride).

In some embodiments, the MAP scaffold has a porosity sufficient to retain the one or more cells that are capable of releasing insulin in response to an elevated glucose concentration (e.g., endocrine precursor cell-derived cells and/or progeny thereof, pancreatic islet cells, and pancreatic islet-like cells derived from pluripotent stem cells (PSCs), optionally induced pluripotent stem (iPS) cells) within the MAP scaffold after introduction of the composition into a subject. In some embodiments, the MAP scaffold has a porosity of about 10 μm to about 200 μm. This porosity serves to retain the pancreatic islet cells, pancreatic islet-like cells derived from induced pluripotent stem (iPS) cells, or the combination thereof within the MAP scaffold after introduction of the composition into a subject but also permit sufficient and rapid vascularization of the cells within the subject.

In some embodiments, the endocrine precursor cell-derived cells and/or progeny thereof, pancreatic islet cells, and pancreatic islet-like cells derived from pluripotent stem cells (PSCs), iPS) cells, and/or the combination are encapsulated within microporous annealed particles (MAPs) per se. Thus, in some embodiments the presently disclosed subject matter relates to compositions comprising, consisting essentially of, or consisting of a single cell suspension comprising, consisting essentially of, or consisting of endocrine precursor cell-derived cells and/or progeny thereof, pancreatic islet cells, and pancreatic islet-like cells derived from pluripotent stem cells (PSCs), optionally induced pluripotent stem (iPS) cells encapsulated by MAPs. In some embodiments, the MAPs comprise a polymer backbone comprising, consisting essentially of, or consisting of poly(ethyleneglycol) (PEG), hyaluronic acid, polyacrylamide, polymethacrylate, alginate, collagen, or any combination thereof. In some embodiments, the MAPs comprise one or more of a PEG-Maleimide, optionally wherein the PEG-Maleimide is a 10 kiloDalton (kDa) PEG-Maleimide; an MMP-2 degradable crosslinker, optionally wherein the MMP-2 degradable crosslinker comprises, consists essentially of, or consists of an amino acid sequence comprising, consisting essentially of, or consisting of Ac-GCGPQGIAGQDGCG-NH₂ (SEQ ID NO: 1); an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer, optionally wherein the annealing monomer is a MethMal annealing macromer.

In some embodiments, a composition of the presently disclosed subject matter comprises, consists essentially of, or consists of a microporous annealed particle (MAP) scaffold and a single cell suspension present therein, wherein the single cell suspension comprises endocrine precursor cell-derived cells and/or progeny thereof, pancreatic islet cells, and pancreatic islet-like cells derived from pluripotent stem cells (PSCs), optionally induced pluripotent stem (iPS) cells; and also a single cell suspension comprising, consisting essentially of, or consisting of endocrine precursor cell-derived cells and/or progeny thereof, pancreatic islet cells, and pancreatic islet-like cells derived from pluripotent stem cells (PSCs), optionally induced pluripotent stem (iPS) cells, or a combination thereof encapsulated by MAPs.

In some embodiments, the endocrine precursor cell-derived cells and/or progeny thereof, pancreatic islet cells, and pancreatic islet-like cells derived from pluripotent stem cells (PSCs), optionally induced pluripotent stem (iPS) cells are isolated and treated as set forth in any of U.S. Pat. Nos. 7,510,876; 7,534,608; 7,541,185; 7,625,753; 7,695,963; 7,695,965; 7,704,738; 7,958,585; 7,993,916; 7,993,920; 8,008,075; 8,129,182; 8,178,878; 8,211,699; 8,216,836; 8,278,106; 8,334,138; 8,338,170; 8,859,286; 8,895,300; and 9,109,245; the entire disclosure of each of which is incorporated herein by reference. D'Amour et al., 2005 describes the production of enriched cultures of human embryonic stem cell-derived definitive endoderm in the presence of a high concentration of activin and low serum and transplanting the same under the kidney capsule of mice, which resulted in differentiation into more mature cells with characteristics of some endodermal organs. D'Amour et al., 2006 discloses a differentiation process that can be used to differentiate human embryonic stem (hES) cells to endocrine cells capable of synthesizing insulin and other hormones.

Human embryonic stem (ES) cell-derived definitive endoderm cells can also differentiated into PDX1 positive cells after addition of FGF-10 (see U.S. Patent Application Publication No. 2005/0266554, which is incorporated herein by reference in its entirety).

U.S. Patent Application Publication No. 2006/0040387 (incorporated herein by reference in its entirety) discloses a system for producing pancreatic islet cells from human embryonic stem cells. As described therein, hES cells were first differentiated to endoderm using a combination of sodium butyrate and activin A. The cells were then cultured with TGF-β antagonists such as Noggin in combination with EGF or betacellulin to generate PDX1 positive cells. Terminal differentiation was induced by nicotinamide. Benvenistry et al., 2006 discloses that over-expression of PDX1 enhances expression of pancreatic enriched genes, but indicates that induction of insulin expression may require additional signals that are only present in vivo (see Benvenistry et al, 2006). Additional references that disclose methods for inducing insulin production in hES cells include Johansson et al., 2007; Diez et al., 2009; Chen et al., 2009 (indolactam V [(ILV)] directs the pancreatic specification of hESCs that have already been committed to the endoderm lineage, including induction of PDX-1 expressing cells; and Lyttle et al., 2008. Finally, U.S. Pat. No. 9,752,125 (incorporated herein by reference in its entirety) discloses an alternative approach to improve the efficiency of differentiating human embryonic stem cells toward insulin expressing cells, by generating a population of cells expressing markers characteristic of the pancreatic endoderm lineage, wherein greater than 50% of the cells in the population co-express PDX-1 and NKX6.1.

Thus, in some embodiments the endocrine precursor cell-derived cells and/or progeny thereof, pancreatic islet-like cells are derived from hES cells and/or are dedifferentiated reprogrammed pluripotent stem cells such as human ES cells and/or human induced pluripotent stem (iPS) cells. Methods for producing pancreatic islet-like cells from dedifferentiated human reprogrammed pluripotent stem cells, such as induced pluripotent stem (iPS) cells, and methods for producing and using such cell culture compositions are known in the art as described in, for example, U.S. Pat. Nos. 8,759,098; 9,365,830 (including a listing of Human Induced Pluripotent Stem (hIPS) Cell Lines and sources thereof); U.S. Pat. Nos. 9,546,379; 9,650,610; 9,982,235; and 11,136,554; each of which is incorporated by reference herein in its entirety. See also Cerf, 2013a; Cerf, 2013b; Migliorini et al., 2021; Jim & Jiang, 2022; Zeynaloo et al., 2022, each of which is incorporated by reference herein in its entirety.

The compositions of the presently disclosed subject matter include degradable linkers/crosslinkers that in some embodiments are designed to release one or more constituents of the compositions after administration to a subject. In some embodiments, the degradable linkers/crosslinkers are thus biodegradable. In some embodiments, a degradable linker/crosslinker of the presently disclosed subject matter is designed to be degraded by matrix metallopeptidase 1 (MMP-1) and/or matrix metallopeptidase 2 (MMP-2) when the composition is administered to a subject. An exemplary MMP-2 degradable linker comprises, consists essentially of, or consists of the amino acid sequence Ac-GCGPQGIAGQDGCG-NH₂ (SEQ ID NO: 1). Other linker sequences that can be degraded by MMP-1 and/or MMP-2 include those listed in Patterson & Hubbell, 2010, which is incorporated herein by reference in its entirety.

In some embodiments, the degradable linker/crosslinker comprises, consists essentially of, or consists of an RGD peptide. In some embodiments, the RGD peptide comprises, consists essentially of, or consists of an amino acid sequence comprising, consisting essentially of, or consisting of the amino acid sequence Ac-RGDSPGGC-NH₂ (SEQ ID NO: 2). In some embodiments, the compositions of the presently disclosed subject matter comprise crosslinkers that are combinations of SEQ ID NOs: 1 and 2 as well as other degradable linkers/crosslinkers.

### II.B. Pharmaceutical Compositions, Methods of Use, and Uses Thereof

In some embodiments, the presently disclosed subject matter also provides methods for treating diabetes (e.g., Type 1 diabetes and/or Type 2 diabetes) in a subject in need thereof. In some embodiments, the methods comprise administering to a subject with diabetes a composition as disclosed herein via a route and in an amount effective for treating the diabetes in the subject. In some embodiments, the administering comprises injecting the composition into a kidney capsule, subcutaneously, intraperitoneally, into adipose tissue, intramuscularly, intrahepatically, and/or intrapancreatically into the subject.

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means. As such, in some embodiments the presently disclosed compositions are administered by injecting the composition into a kidney capsule, subcutaneously, intraperitoneally, into adipose tissue, intramuscularly, intrahepatically, and/or intrapancreatically into the subject.

In some embodiments, a composition of the presently disclosed subject matter is introduced into the kidney capsule of a subject in need thereof. Methods for introducing composition into the kidney capsule of subjects are known, For example, U.S. Pat. No. 9,752,125 (incorporated herein by reference in its entirety) discloses a method wherein a 24G×¾″ I. V. catheter can be used to penetrate the kidney capsule. The catheter can be advanced under the kidney capsule to the distal pole of the kidney. The Pos-D pipette tip was placed firmly in the hub of the catheter and the composition can be dispensed from the pipette through the catheter under the kidney capsule and delivered to the distal pole of the kidney. The kidney capsule can thereafter be sealed with a low temperature cautery before returning the kidney its original anatomical position.

In accordance with some embodiments of the presently disclosed subject matter, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

U.S. Pat. No. 10,273,453 (incorporated herein by reference in its entirety) also discloses a method for introducing biologically active substances into the kidney capsule. This patent discloses that neural crest stem cells (NCSCs) transplanted under the kidney capsule of one pole of the kidney extensively migrate towards co-transplanted pancreatic islets placed in the opposite pole of the same kidney (Olerud et al., 2009). See also U.S. Pat. No. 11,274,280 and U.S. Patent Application Publication No. 2022/0275340.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments, or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion, intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1985; Gennaro, 1990; or Gennaro, 2003; each of which is incorporated herein by reference.

Typically, dosages of the compound of the presently disclosed subject matter which may be administered to an animal, in some embodiments a human, range in amount from 1 ng to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In some embodiments, the pharmaceutical compositions useful for practicing the presently disclosed subject matter may be administered to deliver a dose of between 1 ng/kg/day and 100 mg/kg/day.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a solvent, in some embodiments a sterile solvent, suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Introduction to the EXAMPLES

As disclosed herein, the delivery of dissociated islets (as a model cell type for stem-cell derived beta cells) without pre-implantation conditioning was investigated using microporous annealed particle (MAP) scaffold, which is an injectable biomaterial composed entirely of hydrogel microspheres that are annealed in situ to provide a material environment with cell-scale porosity (Griffin et al., 2015). The MAP scaffold platform has been previously used to accelerate dermal wound healing (Griffin et al., 2015), but has since been shown to be successful in a multitude of other regenerative medicine applications (Nih et al., 2017; Pruett et al., 2020), including serving as a delivery vehicle for stem cells (Koh et al., 2019), conveying anti-inflammatory effects to host tissues through recruitment of pro-regenerative macrophages (Pruett et al., 2021a), and promoting a Th2 “tissue repair” type T-cell response (Griffin et al., 2021).

Microgel production and purification. The no-heparin 3.2 wt % (w/v) microgel precursor solution consisted of PEG-Maleimide (10 kDa, Nippon Oil Foundry, Japan), MMP-2 degradable crosslinker (Ac-GCGPQGIAGQDGCG-NH₂; SEQ ID NO: 1; Watson Bio), RGD (Ac-RGDSPGGC-NH2 (SEQ ID NO: 2); Watson Bio) and the MethMal heterofunctional maleimide/methacrylamide 4-arm PEG macromer (MethMal; see Pfaff et al., 2021) annealing macromer. The 2.2 wt % 6 mg/mL heparin microgel precursor solution contained thiolated heparin which was prepared as described in Pruett et al., 2021a. Microgels were synthesized using a high-throughput microfluidics technique as described in Roosa et al., 2022. The aqueous phase was run at 3 mL/hr and the surfactant solution (2% Pico-Surf in NOVEC 7500) was run at 6 mL/hr through the microfluidic device, and microgels were collected in a 50 mL conical tube. An oil solution with 3% vol/vol triethylamine was added to the microgel suspension to increase the pH and initiate microgel crosslinking. After complete gelation, microgel purification and sterilization was performed as described in Pruett et al., 2021a. For animal studies, heparin-containing microgels were mixed in with the no-heparin microgels at a 1:10 ratio (10% heparin-islands). Prior to annealing, microgels were mixed 1:1 with a 0.2 mM lithium phenyl-2,4,6-trimethylbenzoylphosphinate (LAP) solution for cell studies or mixed 1:1 with a 40 μM Eosin Y solution for animal studies.

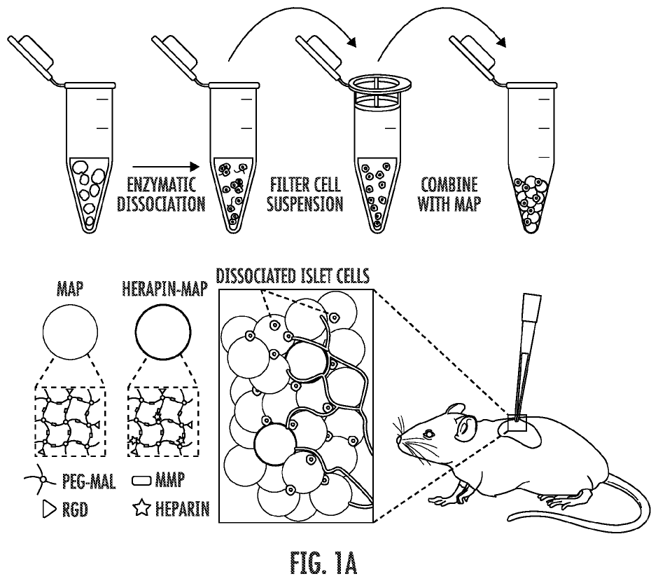

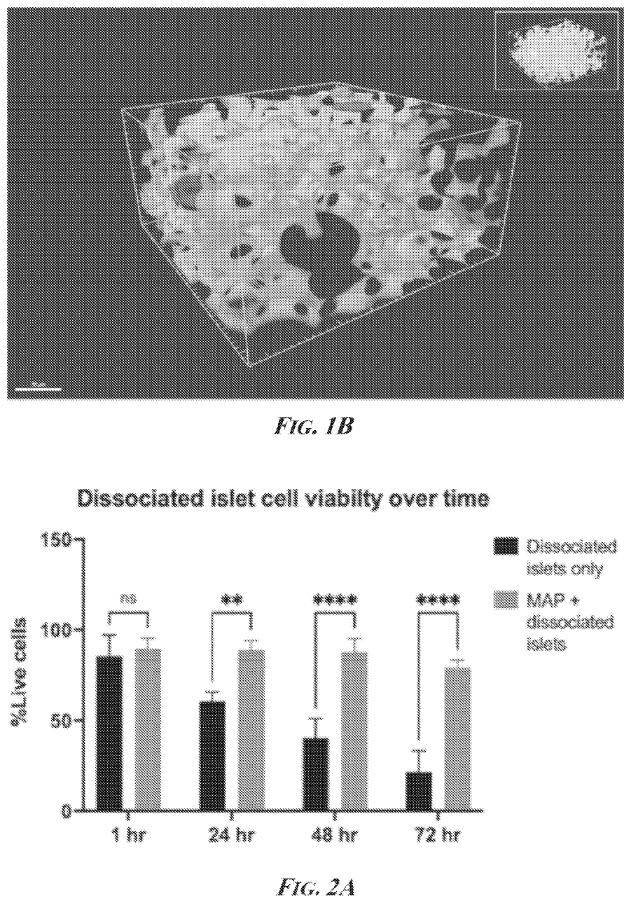

Pore sizing. Heparin-containing microgels (labeled with TexasRed fluorophore) were mixed in with the no-heparin microgels at a 1:10 ratio (10% heparin-μislands) and incubated with 0.2 mM LAP for 15 minutes. Next, three 5 μL pucks of gel were added to a glass slide with a 1 mm-thick spacer and annealed with 365 nm LED light for 30 seconds. A 300 μg/mL dextran solution (Oregon Green 488, 70 kDa) was added on top of each gel puck and allowed to diffuse into the pores for at least 30 minutes prior to imaging. A Zeiss 710 Laser Scanning Microscope was used in 2-Photon mode to image at least a 200 μm z-stack (1 μm or 10 μm step-size) through each scaffold. (n=3) The z-stacks from three different scaffolds were then exported to Imaris Microscopy Image Analysis software (Oxford Instruments) to produce 3D rendering of the pores (green dextran) and heparin-μislands particles (red) (FIG. 1B). Imaris software was also used to threshold the area of the green dextran (pores) in each slice and the median pore area was calculated.

Isolation and dissociation of pancreatic islets. Islets were isolated by collagenase digestion and density gradient centrifugation. Under anesthesia, the pancreas was exposed by midline laparotomy, the common bile duct identified and cannulated, and the pancreas distended [collagenase P, 2.5 ml, 2 mg/ml in Hank's balanced salt solution (HBSS); Roche Molecular Biochemicals, Indianapolis, IN]. The distended pancreas was excised, digested (13 min, 37° C.), and shaken vigorously to mechanically disrupt the tissue (30 s). Digestion was quenched [excess cold HBSS with 10% fetal calf serum (FCS); Invitrogen, Grand Island, NY], and the digested tissue filtered through a nylon mesh (1,000 μm, Nitex; Fisher Scientific) and washed (3×, HBSS, 0° C.). Islets were purified by density gradient centrifugation using Histopaque 1077 (Sigma-Aldrich, St. Louis, MO). Islets were washed and cultured (˜18 h, 37° C., 5% CO2) in DMEM (Invitrogen) supplemented with 10% FCS, 2 mM 1-glutamine, 1% penicillin/streptomycin, 0.1 mM MEM nonessential amino acids, and 25 mM HEPES buffer (all supplements from Invitrogen; 20 mL per plate). Islets were then hand-picked and transferred to sterile Eppendorf tubes and washed with PBS twice following which islets were resuspended in 300 μL of 0.05% Trypsin-EDTA (Gibco) and incubated in a 37° C. water bath for 10 minutes. The tube was gently tapped every 2 min to mechanically dissociate cells. 300 μL of DMEM media with 10% FCS was added to the cells to neutralize the trypsin-EDTA. The cell suspension was filtered with a 35 μm cell strainer to isolate single cells and remove residual extracellular matrix. Dissociated cells were centrifuged at 0.2×1000 g for 5 minutes and supernatant carefully removed. The cell pellet was resuspended with fresh DMEM media.

Viability assay. Cell survival was determined using fluorescein diacetate (FDA, Sigma) and propidium iodide (PI, Sigma) live/dead stains. For each condition, 50 islets were dissociated with 0.05% trypsin-EDTA, filtered, and the resulting cell suspension was mixed with 80 μL of MAP gel or 80 μL of media and transferred to a 24 transwell plate insert. Wells received 1 mL of DMEM media and the plate was incubated at 37° C. in between timepoints. At each timepoint (1, 24, 48, and 72 hours), the transwell inserts (n=3 per timepoint) were removed from media and placed into 1 mL of PBS containing 50 ng/ml of FDA and 14.5 μg/mL of PI. After incubation for 30 seconds with gentle shaking at room temperature, the transwell was removed and excess moisture was removed by gently blotting bottom of membrane with a Kimwipe. Wells were imaged with 4× objective on an EVOS FL Auto microscope. Images were imported into ImageJ, and the red (dead) and green (live) channels were thresholded and cells were counted using the ImageJ Particle Analysis plugin. Cell viability is reported as:

[(sum of live cells)/(sum of live+dead cells)]×100%.

IHC staining. Kidneys containing the graft were excised at the study endpoint and were immediately fixed in formalin. Prior to paraffin embedding, kidneys were cut in half and the half containing the graft was submitted to the University of Virginia Histology Core. Samples were embedded in paraffin and sectioned transversely with a microtome into sections with 5 μm thickness. Immunohistochemistry was performed by the University of Virginia Biorepository and Tissue Research Facility (BTRF). Recombinant Anti-Insulin antibody (rabbit monoclonal [EPR17359] to insulin) was purchased from Abcam (Catalog No. ab 181547) and optimized by the BTRF. Positive-staining was confirmed by dark-brown DAB chromogen stain.

Statistical Analysis. Statistical analysis was performed in GraphPad Prism software. An ordinary two-way ANOVA with Sidak's multiple comparison test (single pooled variance) was used to compute differences between groups in the cell viability and GSIS data. An ordinary one-way ANOVA with Tukey's multiple comparison test was used to compute differences between groups in the dissociated islet cell density viability experiment.

### Example 1

### MAP as a Delivery Platform for Dissociated Islet Cells

A recently designed MAP scaffold formulation comprising a heterogenous composition that adds heparin-containing microspheres (heparin μislands), which has previously demonstrated the ability to organize endogenous growth factors in a diabetic wound environment and significantly improve re-vascularization (Pruett et al., 2021a; Pruett et al., 2021b), was tested. Given the success using MAP scaffold as an angiogenic and immunomodulatory biomaterial capable of cell delivery, MAP was selected as a delivery platform for dissociated islet cells (containing beta, alpha, and delta cells) to treat TID in mice. By mixing dissociated islet cells with MAP and assembling the scaffold in situ (FIG. 1A), it was possible to support cell organization, survival, and function for a prolonged period, which did not require prior in vitro formation of pseudoislets.

The present investigation focused on a MAP microgel formulation that consisted of a 4-arm polyethylene glycol (PEG)-maleimide backbone, an enzymatically degradable peptide crosslinker, a cell adhesive peptide pendant group, and a custom MethMal annealing macromer (previously described for accelerated MAP assembly in situ; see Pfaff et al., 2021). For animal studies, a minority of microgels containing immobilized thiolated heparin were included heterogeneously throughout the MAP scaffold to promote accelerated vascularization (Pruett et al., 2021a). All microgels were synthesized using a high-throughput microfluidics technique as described herein above (Microgel production and purification; see also de Rutte et al., 2019). After separate microgel synthesis and purification, heparin-containing microgels were mixed in with the no-heparin microgels at a 1:10 ratio (10% heparin-μislands). This ratio was chosen based on previously published data, where 10% heparin-μislands facilitated significantly increased endothelial cell behavior in vitro and in vivo (Pruett et al., 2021a). To visualize scaffold porosity, 200 μm thick MAP scaffolds containing 10% heparin islands were incubated with fluorescent dextran and imaged with a Zeiss 710 laser scanning confocal microscope (FIG. 1B; see Pore sizing herein above). The median pore area of the scaffolds (n=3) was 526.4 μm², which is equivalent to 25.9 μm diameter circular pore.

### Example 2

### Impact of MAP Scaffold on Dissociated Islet Cell Viability In Vitro

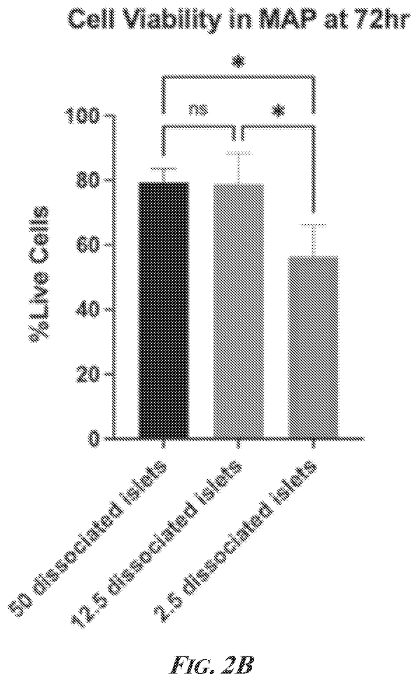

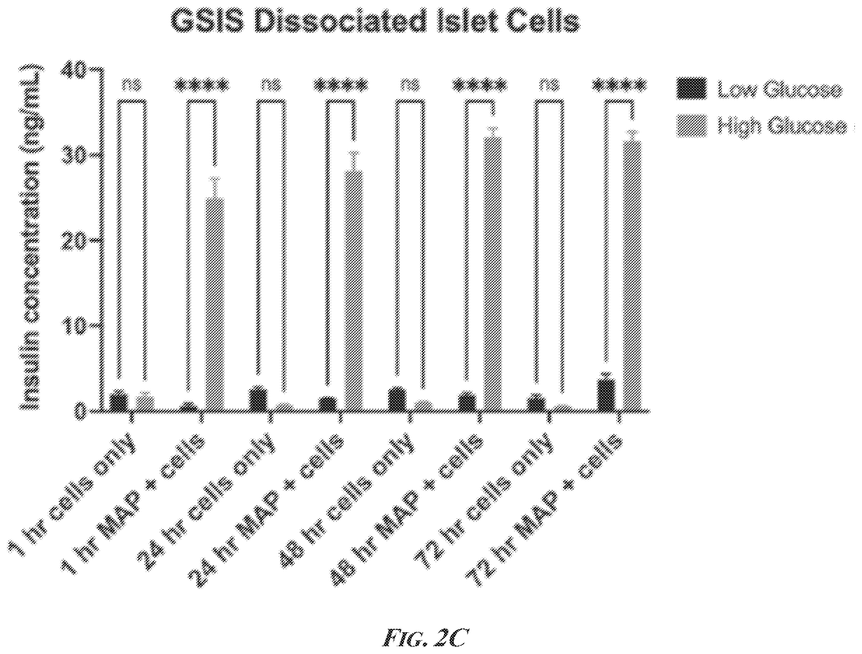

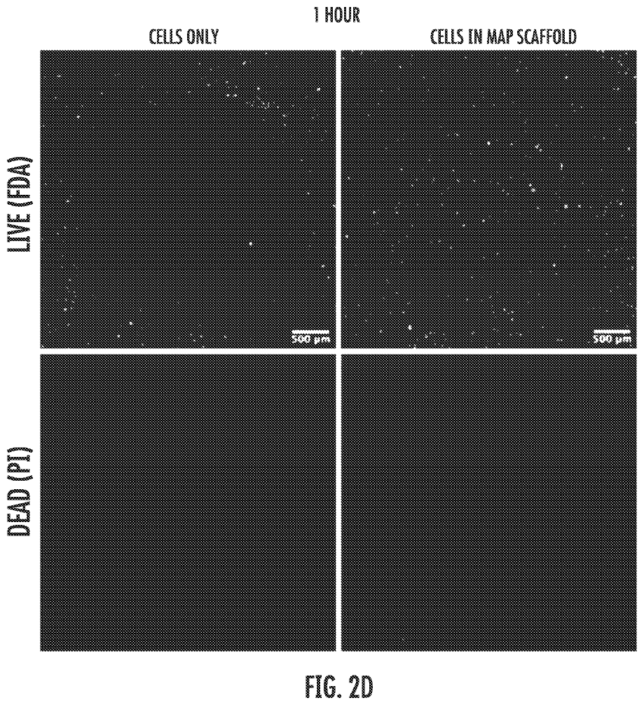

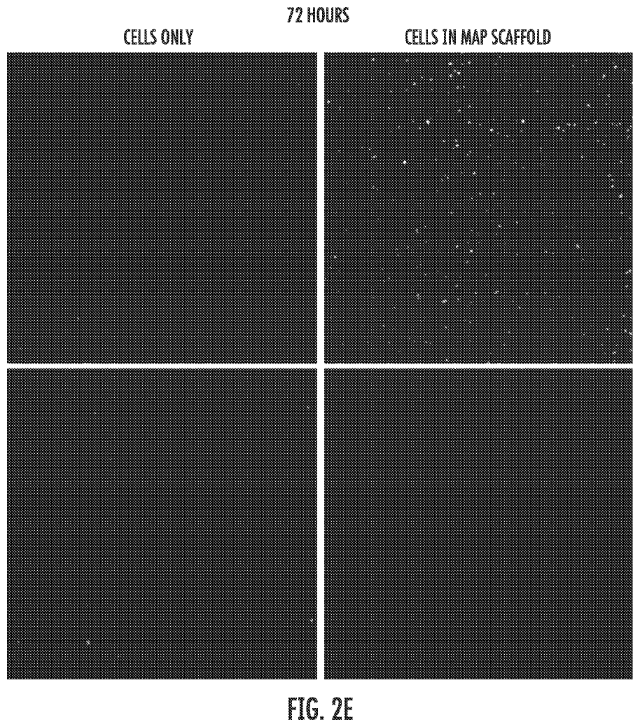

The impact of MAP scaffold on dissociated islet cell viability was first assessed in vitro. Islets harvested from healthy donor C57BL/6 mice were isolated by collagenase digestion and density gradient centrifugation as described herein above (see Isolation and dissociation of pancreatic islets). Isolated islets were resuspended in 300 μL of 0.05% Trypsin-EDTA and incubated in 37° C. water bath for 10 minutes with tapping to mechanically dissociate the islet clusters. The resulting digestate was filtered with a 35 μm cell strainer to isolate a single cell suspension. The MAP scaffold was sterilized and mixed 1:1 with a 0.2 mM lithium phenyl-2,4,6-trimethylbenzoylphosphinate (LAP) photoinitiator solution. For each condition, 50 islets were dissociated, filtered, and the resulting cell suspension was mixed with 80 μL of MAP gel or 80 μL of media and transferred to a 24 transwell plate insert. The MAP scaffold was annealed with a 365 nm LED light (ThorLabs Inc., Newton, New Jersey, United States of America) for 30 seconds. At each timepoint (1, 24, 48, and 72 hours), the transwell inserts (n=3 per timepoint) were removed from media and placed into 1 mL of PBS containing 50 ng/ml of fluoresceine diacetate (live cell stain) and 14.5 μg/mL of propidium iodide (dead cell stain). Wells were imaged with 4× objective on an EVOS FL Auto microscope and images were analyzed in ImageJ (see Viability Assay herein above). After 1 hour, there was a slight decline in cell viability in both conditions (˜10% loss in viability), likely due to cell death that occurred during the dissociation process (FIG. 2A). After 24 hours, the cell viability in the cells only condition had decreased significantly (60% live) compared to MAP+cells condition (89% live). Cell viability in cells only condition continued to decline after 48 hours (38% live) and 72 hours (21% live) incubation. The dissociated islet cells mixed with MAP scaffold maintained significantly higher viability after 48 hours (87% live) and 72 hours (79% live) incubation time periods. It is notable that MAP scaffold facilitates dissociated islet cell survival up to 72 hours in vitro (FIG. 2D).

### Example 3

### Effects of MAP Scaffold on Different Concentrations of Dissociated Islets and Assessed Cell Viability at 72 Hours

Based on previously published data that dissociated islet viability is dependent on cell-packing density (Lin et al., 2011), the effects of MAP scaffold on three different concentrations of dissociated islets and assessed cell viability at 72 hours was investigated. Dissociated cells from 50, 12.5, or 2.5 islets (approximately 2,000 cells/islet) were mixed with MAP as described above, and incubated in a transwell plate for 72 hours (n=3 per condition). Cell viability in the 50 and 12.5 dissociated islet conditions was on average >78%, and there was no significant difference between groups (FIG. 2B). However, the dissociated cells from 2.5 islets were only 56% viable after 72 hours, which was a significant decrease compared to the other two conditions. This indicates that even in MAP scaffold, dissociated islet cell viability is still density dependent.

### Example 4

### Glucose Stimulated Insulin Secretion (GSIS) Assay

The effects of MAP scaffold on dissociated islet cell function were further evaluated using a glucose stimulated insulin secretion (GSIS) assay. For each condition, 30 islets were dissociated, filtered, and the resulting cell suspension was mixed with 80 μL of sterile MAP scaffold (no-heparin) or 80 μL of media and transferred to a 24 transwell plate insert. At each timepoint (1, 24, 48, and 72 hours), the transwell inserts (n=3 per timepoint) were removed from media and placed into Krebs-Ringer bicarbonate (KRB) buffer supplemented with low (2.8 mM) glucose for 1 hour. Then, the transwell inserts were transferred to high (28 mM) glucose buffer for 1 hour. The buffer from low and high glucose conditions was collected and assessed by insulin ELISA (mouse insulin ELISA kit, Mercodia Inc., Winston-Salem, North Carolina, United States of America). It was observed that the cells within the MAP scaffold retained their glucose-sensing abilities, and secreted significantly higher amounts of insulin in response to the high-glucose environment compared to the low glucose environment (FIG. 2C). The cells seeded without MAP scaffold exhibited a significant decrease in function, even after 1 hour incubation. There was no significant difference in insulin secretion between high glucose and low glucose conditions at any timepoint for the cells only condition. This indicates that dissociated islet cells lose their glucose-sensing capabilities and are unable to secrete insulin in response to glucose challenge even after 1 hour incubation on a 2D plastic substrate. Thus indicating that the presently disclosed MAP scaffold offered the environment needed to maintain beta cell function.

### Example 5

### Efficacy of MAP Delivery of Dissociated Islet Cells in a Syngeneic Mouse Model of Diabetes

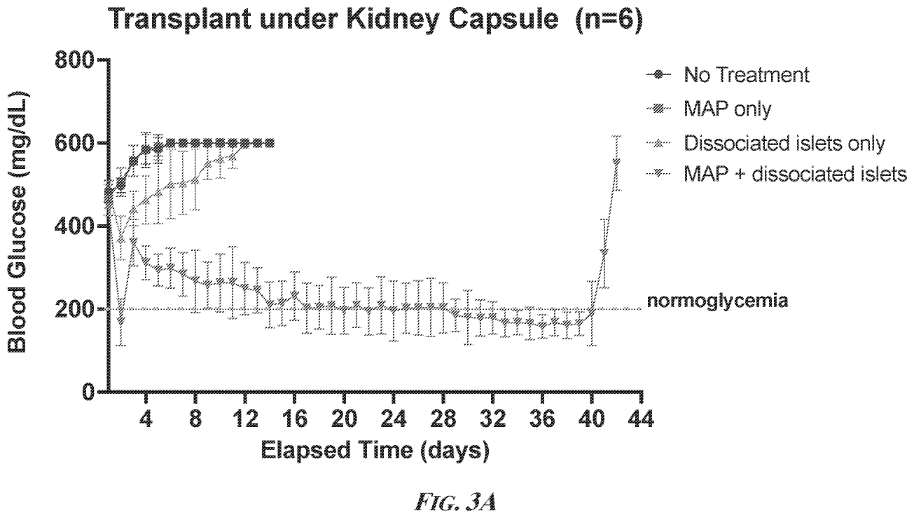

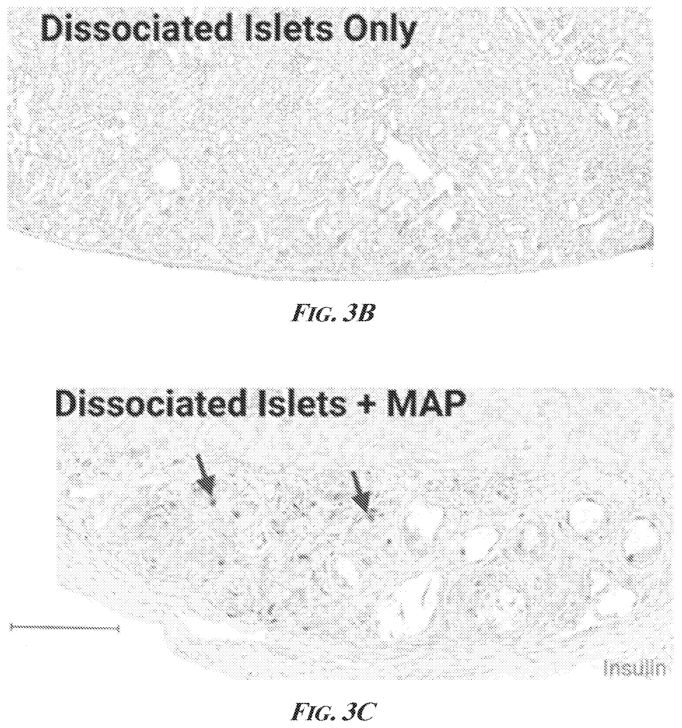

Following the positive in vitro results described above, the efficacy of MAP delivery of dissociated islet cells in a syngeneic mouse model of diabetes was investigated. Recipient C57BL/6 mice were rendered diabetic by a single intraperitoneal injection of streptozotocin (STZ, 250 mg/kg). For transplantation at the renal subcapsular site, diabetic recipient mice were anesthetized, and the left kidney exposed through the left flank. 100 islets harvested from C57BL/6 donors were dissociated and mixed with 15 μL of MAP scaffold (containing 10% heparin-μislands) and injected through a small incision in the renal capsule using a positive displacement pipette. Control animals received 15 μL of MAP scaffold alone. The scaffold was annealed in situ by exposing the injection site to a 505 nm LED light (ThorLabs Inc.) for 2 minutes. For control groups, dissociated cells from 100 islets were injected directly under renal capsule (no MAP) or the animals received no treatment. The kidney was returned to anatomical position, and the skin and muscle closed separately. Recipient blood glucose levels (non-fasting) were monitored daily (AccuChek blood glucose meter; Roche Diabetes Care, Inc., Indianapolis, Indiana, United States of America), and diabetes was defined as blood glucose (BG)>300 mg/dl on 2 consecutive days, with cure defined as a return to normoglycemia (BG<200 mg/dl for 2 consecutive days). Blood glucose levels of recipient mice were monitored every day for up to 44 days. By day 16, the mice that received MAP only, dissociated islets only, or no treatment had achieved hyperglycemic blood glucose levels (>600 mg/dL) and were sacrificed in accordance with a protocol approved by the Institutional Animal Care and Use Committee of the University of Virginia (Charlottesville, Virginia, United States of America). By day 17, the mice that received MAP mixed with dissociated islets achieved normoglycemia (<200 mg/dL) and maintained normal glucose levels for up to 40 days post-transplant (FIG. 3A). Left nephrectomy at day 40 caused a spike in blood glucose, which indicated that the implant itself was regulating the blood glucose. The dissociated islet cells implanted with MAP scaffold retained their ability to secrete insulin and maintain normoglycemia in a syngeneic implant model of STZ-induced diabetes, whereas the cells alone did not. Kidneys removed at the study endpoint were fixed, sectioned, and stained with anti-Insulin antibody (see IHC Staining herein above). Immunohistochemistry revealed positive insulin staining of beta cells in the MAP implant (day 40 timepoint; FIG. 3C), whereas no positive staining is seen in the implant site of the cells only transplant (FIG. 3B).

### Example 6

### Encapsulation of Cells in MAPs

In some embodiments of the presently disclosed subject matter, cells, optionally single cell suspensions of pancreatic islet cells, pancreatic islet-like cells derived from induced pluripotent stem (iPS) cells, or combinations thereof, were present not only within MAP scaffolds but also encapsulated by microgels of microporous annealed particles (MAPs) per se. These microgels were made as described herein with the exception that the cells are included in the mixture when the microgel was created. An exemplary photomicrograph of a microgel of MAPs encapsulating cells is presented in FIG. 4.

### Example 7

### Immunomodulatory Effects of MAP Scaffold for Beta Cell Delivery to Fat Tissue

The epididymal fat pad in mice is a more clinically relevant and less immune-privileged transplant site than the kidney capsule. MAP scaffold and nanoporous (NP) gels were transplanted into the fat pad of non-diabetic mice with or without beta cells, and the cytokine profile of the implants were analyzed by a multiplex proinflammatory cytokine assay after 7 days post-transplant. A commercially available beta cell line, Beta TC-6 (ATCC), was cultured according to protocol. The MAP scaffold and NP gel formulation both consisted of a 3.2 wt % (w/v) gel precursor solution of PEG-Maleimide (10 kDa, Nippon Oil Foundry, Japan), MMP-2 degradable crosslinker (Ac-GCGPQGIAGQDGCG-NH₂; SEQ ID NO: 1; Watson Bio), RGD (Ac-RGDSPGGC-NH₂; SEQ ID NO: 2; Watson Bio) and MethMal (Pfaff et al., 2021) annealing macromer. Recipient C57BL/6 mice (male retired breeders) were anesthetized and placed in the supine position. Hair at the incision site was removed using a razor, and surgical scissors were used to create an incision in the inguinal region near the midline. The fat pad was carefully removed with forceps and spread out onto the abdomen.

After the treatment was applied, the fat pad was carefully folded and sutured close to form a “pocket”. Then, the fat pad was placed back into position and the skin and muscle were sutured closed. 1×10⁶ Beta TC-6 cells were mixed with 15 μL of MAP scaffold or encapsulated within 10 μL NP gels and transplanted into the fat pad of non-diabetic mice (n=5 per group), and the implants were removed after 7 days. After the implants were removed, they were stored at −80° C.

To process the implants for the multiplex proinflammatory cytokine assay, the implants were thawed at RT and mixed with 0.5 mL of cell lysis buffer (Cell Signaling Technologies), then homogenized thoroughly with a mechanical tissue homogenizer. The tissue lysate was spun down at 250,000×g, and the supernatant was collected and submitted for analysis with a proinflammatory cytokine panel (Luminex). The cytokine protein concentration (pg/mL) was normalized to the total protein concentration in each sample (mg/mL).

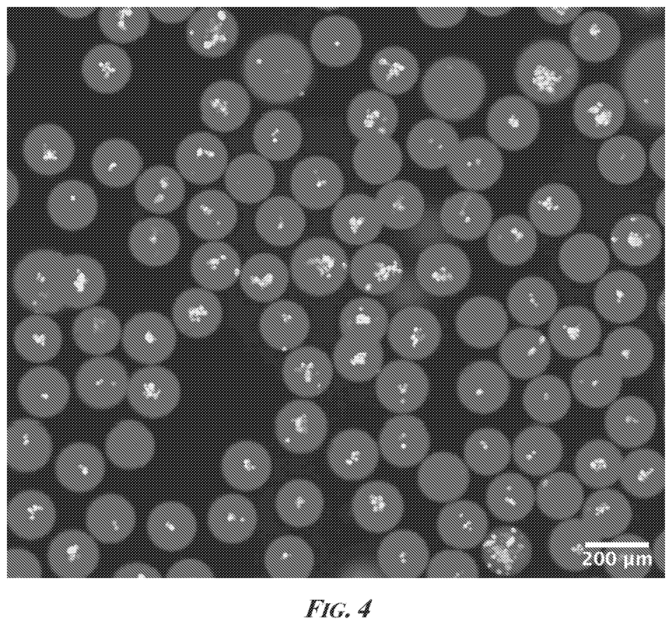

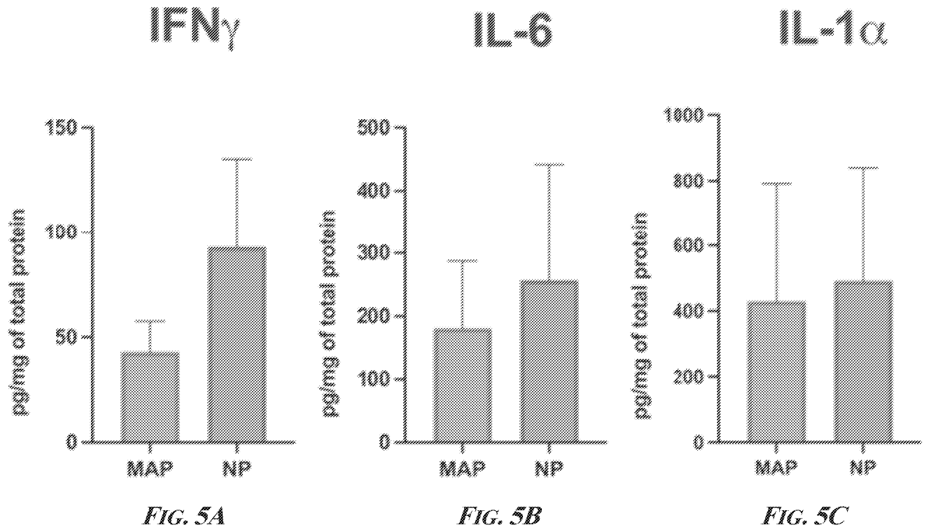

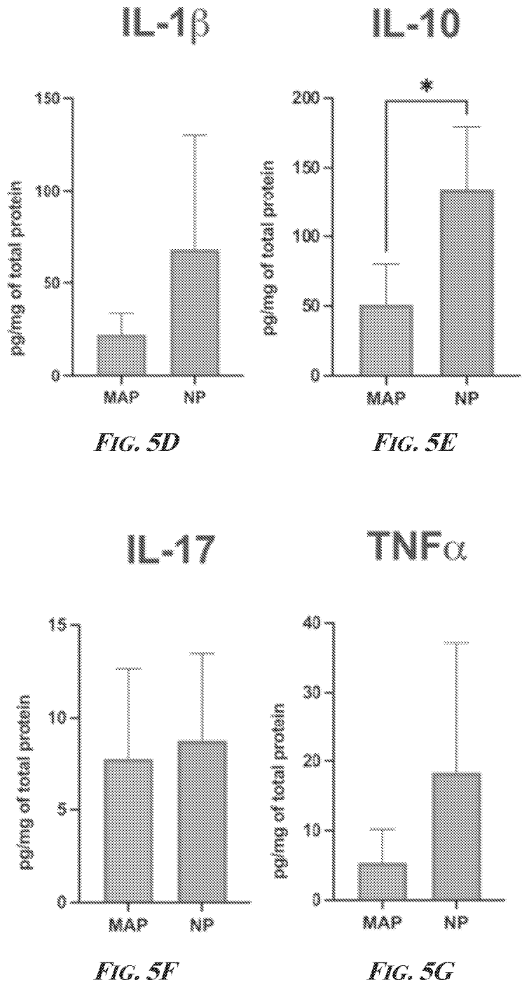

The results are shown in FIGS. 5A-5G. The cytokines assayed were interferon gamma (IFNγ; FIG. 5A), interleukin-6 (IL-6; FIG. 5B), interleukin-1 alpha (IL-1α; FIG. 5C), interleukin-1 beta (IL-1B; FIG. 5D), interleukin-10 (IL-10; FIG. 5E), interleukin-17 (IL-17; FIG. 5F), and tumor necrosis factor alpha (TNFα; FIG. 5G). As shown in FIGS. 5A-5G, there appeared to be a strong trend which suggested that MAP scaffold down-regulated some of the pro-inflammatory cytokines at the implant site, which was likely important to ensure the survival of the transplanted beta cells. These data also indicated that MAP had immunomodulatory effects on the transplant site, including a significant decrease in IL-10, a canonical inflammatory cytokine, and a trending decrease across the remainder of the cytokine panel.

### Discussion of the EXAMPLES

In summary, presented herein is the successful use of a unique injectable biomaterial, microporous annealed particle (MAP) scaffold, as a platform for delivery of dissociated islet cells. Specifically, it has been demonstrated that MAP scaffold maintains dissociated islet cell survival and function (i.e., glucose-sensing and insulin secretion) in vitro for at least 3 days and in vivo for up to 40 days post-transplant. The specific mechanisms by which MAP scaffold may support function of single cell suspensions of beta cells (e.g., immunomodulation, enhanced angiogenesis, cellular pathways, etc.) will be investigated in future studies. Importantly, this is the first study that demonstrates the feasibility of delivering dissociated islets within MAP scaffold to re-establish normoglycemia in a model of TID. These findings may lead to translational approaches that will address the scarcity of donor pancreatic tissue needed to treat patients suffering from TID. This disclosure thus provides a first step to MAP scaffold-assisted delivery of clinically scalable sources of insulin-secreting cells (e.g., iPSC-derived beta cells).

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Benninger & Piston (2014) Cellular communication and heterogeneity in pancreatic islet insulin secretion dynamics. Trends in Endocrinology and Metabolism 25:399-406. Benvenistry et al. (2006) The effect of overexpression of Pdx1 and Foxa2 on the differentiation of human embryonic stem cells into pancreatic cells. Stem Cells 24:1923-1930.
Bird et al. (1988) Single-chain antigen-binding proteins. Science 242:423-426
CDC (2020) National Diabetes Statistics Report 2020. Estimates of Diabetes and Its Burden in the United States.
Cerf (2013a) Beta cell dynamics: beta cell replenishment, beta cell compensation and diabetes. Endocrine 44(2):303-311.
Cerf (2013b) Beta cell dysfunction and insulin resistance. Front Endocrinol (Lausanne) 4:37.
Chen et al. (2009) A small molecule that directs differentiation of human ESCs into the pancreatic lineage. Nature Chemical Biology 5(4):258-265.
Chhabra & Brayman (2018) Stem cell strategies to promote islet transplantation outcomes. OBM Transplantation 2(2). doi: 10.21926/obm.transplant.1802010.
Collaborative Islet Transplant Registry (2015) Scientific Summary of the Collaborative Islet Transplant Registry (CITR) 2015 (Tenth) Annual Report BACKGROUND AND PURPOSE.
D'Amour et al. (2005) Efficient differentiation of human embryonic stem cells to definitive endoderm. Nature Biotechnology 23:1534-1541.
de Rutte et al. (2019) Scalable high-throughput production of modular microgels for in situ assembly of microporous tissue scaffolds. Advanced Functional Materials 29:190071.
Devereux et al. (1984) A comprehensive set of sequence analysis programs for the VAX. Nucl Acids Res 12:387.
Diez et al. (2009) Endocrine cell clustering during human pancreas development. J Histochem Cytochem 57(9):811-824.
Gao et al. (2011) The Generation of Definitive Endoderm from Human Embryonic Stem Cells on 3D Biodegradable Poly(Lactic-Co-Glycolic Acid) Scaffolds and Its Comparison to Those Generated on 2D Monolayer Cultures. The Open Stem Cell Journal 3:23-27.
Gao et al. (2016) Engineering of microscale three-dimensional pancreatic islet models in vitro and their biomedical applications. Critical Reviews in Biotechnology 36:619-629.
Gennaro (ed.) (1985) Remington's Pharmaceutical Sciences, Mack Publishing Co., Easton, Pennsylvania, United States of America.
Gennaro (ed.) (1990) Remington's Pharmaceutical Sciences, 18th ed., Mack Pub. Co., Easton, Pennsylvania, United States of America,
Gennaro (ed.) (2003) Remington: The Science and Practice of Pharmacy, 20th edition Lippincott, Williams & Wilkins, Philadelphia, Pennsylvania, United States of America.
Griffin et al. (2015) Accelerated wound healing by injectable microporous gel scaffolds assembled from annealed building blocks. Nature Materials 14:737-744.
Griffin et al. (2021) Activating an adaptive immune response from a hydrogel scaffold imparts regenerative wound healing. Nature Materials 20:560-569.
Gross & Mienhofer (eds.) (1981) The Peptides, Vol. 3, Academic Press, New York, New York, United States of America. pages 3-88.
Harlow & Lane, 1988, Antibodies, A Laboratory Manual, Cold Spring Harbor Publications, Cold Spring Harbor, New York, United States of America.
Hilderink et al. (2015) Controlled aggregation of primary human pancreatic islet cells leads to glucose-responsive pseudoislets comparable to native islets. Journal of Cellular and Molecular Medicine 19:1836-1846.
Huston et al. (1988) Protein engineering of antibody binding sites: recovery of specific activity in an anti-digoxin single-chain Fᵥ analogue produced in Escherichia coli. Proc Natl Acad Sci USA 85:5879.
Jin & Jiang (2022) Stepwise differentiation of functional pancreatic β cells from human pluripotent stem cells. Cell Regeneration 11(1):24.
Johansson et al. (2007) Temporal control of neurogenin3 activity in pancreas progenitors reveals competence windows for the generation of different endocrine cell types. Developmental Cell 12:457-465.
Jones et al (1986) Replacing the complementarity-determining regions in a human antibody with those from a mouse. Nature 321:522.
Karlin & Altschul (1990) Methods for assessing the statistical significance of molecular sequence features by using general scoring schemes. Proc Natl Acad Sci USA 87(6):2264-2268.
Karlin & Altschul (1993) Applications and statistics for multiple high-scoring segments in molecular sequences. Proc Natl Acad Sci USA 90(12):5873-5877.
Kodama et al. (2009) Engineering Functional Islets from Cultured Cells. Tissue Engineering—Part A 15:3321-3329.
Koh et al. (2019) Enhanced in vivo delivery of stem cells using microporous annealed particle scaffolds. Small 15:1903147.
Lebreton et al. (2019) Insulin-producing organoids engineered from islet and amniotic epithelial cells to treat diabetes, Nature Communications 10: Article 4491.
Li (2020) Functional analysis of islet cells in vitro, in situ, and in vivo. Seminars in Cell and Developmental Biology 103:14-19.
Lin & Anseth (2009) Glucagon-like peptide-1 functionalized PEG hydrogels promote survival and function of encapsulated pancreatic beta-cells. Biomacromolecules 10:2460-2467.
Lin et al. (2011) Cell-cell communication mimicry with poly(ethylene glycol) hydrogels for enhancing beta-cell function. PNAS 108:6380-6385.
Lyttle et al. (2008) Transcription factor expression in the developing human fetal endocrine pancreas. Diabetologia 51(7):1169-1180.
Migliorini et al. (2021) Human pluripotent stem cell-derived insulin-producing cells: A regenerative medicine perspective. Cell Metab 33(4):721-731.
Nakayama-Iwatsuki et al. (2021) Fabrication of functional rat pseudo-islets after cryopreservation of pancreatic islets or dispersed islet cells. Journal of Tissue Engineering and Regenerative Medicine 15:686-696.
Navarro-Tableros et al. (2004) Autocrine regulation of single pancreatic beta-cell survival. Diabetes 53:2018-2023.
Nih et al. (2017) Injection of Microporous Annealing Particle (MAP) Hydrogels in the Stroke Cavity Reduces Gliosis and Inflammation and Promotes NPC Migration to the Lesion. Advanced Materials 29:100.1002/adma.201606471.
O'Berg-Welsh (2001) Long-term culture in matrigel enhances the insulin secretion of fetal porcine islet-like cell clusters in vitro. Pancreas 22:157-163.
O'Sullivan et al. (2010) Rat islet cell aggregates are superior to islets for transplantation in microcapsules. Diabetologia 53:937-945.
Olerud et al. (2009) Neural crest stem cells increase beta cell proliferation and improve islet function in co-transplanted murine pancreatic islets. Diabetologia 2009; 52:2594-601.
Patterson & Hubbell (2010) Enhanced proteolytic degradation of molecularly engineered PEG hydrogels in response to MMP-1 and MMP-2. Biomaterials 31:7836e7845.
Paul (1993) Fundamental Immunology, 3rd Ed., Raven Press, New York, New York, United States of America.
Pfaff et al. (2021) Selective and Improved Photoannealing of Microporous Annealed Particle (MAP) Scaffolds. ACS Biomaterials Science and Engineering 7:422-427.
Pruett et al. (2020) Development of a microporous annealed particle hydrogel for long-term vocal fold augmentation. Laryngoscope 130:2432-2441.
Pruett et al. (2021a) Heparin Microislands in Microporous Annealed Particle Scaffolds for Accelerated Diabetic Wound Healing. Advanced Functional Materials 31:2104337.
Pruett et al. (2021b) Spatially heterogeneous epidermal growth factor release from microporous annealed particle (MAP) hydrogel for improved wound closure. Journal of Materials Chemistry B 9:7132-7139.
Riechmann et al. (1988) Reshaping human antibodies for therapy. Nature 332(6162):323-327.
Roosa et al. (2022) Microfluidic Synthesis of Microgel Building Blocks for Microporous Annealed Particle Scaffold. J Vis Exp (184): e64119. Doi: 10.3791/64119.
Suarez-Pinzon et al. (2005) Combination therapy with epidermal growth factor and gastrin induces neogenesis of human islet β-cells from pancreatic duct cells and an increase in functional-cell mass. Journal of Clinical Endocrinology and Metabolism 90:3401-3409.
Tran et al. (2020) Developmentally-Inspired Biomimetic Culture Models to Produce Functional Islet-Like Cells From Pluripotent Precursors. Frontiers in Bioengineering and Biotechnology 8:583970.
Tran et al. (2020) Developmentally-Inspired Biomimetic Culture Models to Produce Functional Islet-Like Cells From Pluripotent Precursors. Front Bioeng Biotechnol 8:583970.
Tsang et al. (2007) Generation of Functional Islet-Like Clusters After Monolayer Culture and Intracapsular Aggregation of Adult Human Pancreatic Islet Tissue. Cell Therapy and Islet Transplantation 83:685-693.
U.S. Patent Application Publication Nos. 2003/0017534, 2005/0266554, 2006/0040387, 2012/0039955, 2018/0298087, 2018/0312588, 2018/0346564, 2019/0151448, 2022/0275340.
U.S. Pat. Nos. 4,816,567; 5,482,856; 6,479,284; 6,677,436; 7,060,808; 7,510,876; 7,534,608; 7,541,185; 7,625,753; 7,695,963; 7,695,965; 7,704,738; 7,906,625; 7,958,585; 7,993,916; 7,993,920; 8,008,075; 8,129,182; 8,178,878; 8,211,699; 8,216,836; 8,278,106; 8,334,138; 8,338,170; 8,398,980; 8,436,150; 8,759,098; 8,796,439; 8,859,286; 8,859,286; 8,895,300; 9,109,245; 9,365,830; 9,546,379; 9,650,610; 9,752, 125; 9,982,235; 10,253,111; 10,273,453; 11,136,554; 11,274,280.
Wang & Rosenberg (1999) Maintenance of beta-cell function and survival following islet isolation requires re-establishment of the islet-matrix relationship. Journal of Endocrinology 163:181-190.
Winter & Milstein (1991) Man-made antibodies. Nature 349(6307):293-299.
Youngblood et al. (2019) Microporous scaffolds support assembly and differentiation of pancreatic progenitors into B-cell clusters. Acta Biomaterialia 96:111-122.
Yu et al. (2018) Bioengineered human pseudoislets form efficiently from donated tissue, compare favourably with native islets in vitro and restore normoglycaemia in mice. Diabetologia 61:2016-2029.
Zeynaloo et al. (2022) Delivery of therapeutic agents and cells to pancreatic islets: Towards a new era in the treatment of diabetes. 83 Molecular Aspects of Medicine 101063.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising a microporous annealed particle (MAP) scaffold and a single cell suspension present therein, wherein the single cell suspension comprises, consists essentially of, or consists of one or more cells that are capable of releasing insulin in response to an elevated glucose concentration.

2. The composition of claim 1, wherein the MAP scaffold comprises a polymer backbone comprising, consisting essentially of, or consisting of poly(ethyleneglycol) (PEG), hyaluronic acid, polyacrylamide, polymethacrylate, alginate, collagen, or any combination thereof.

3. The composition of claim 1, wherein the MAP scaffold comprises one or more of a PEG-Maleimide, optionally wherein the PEG-Maleimide is a 10 kiloDalton (kDa) PEG-Maleimide; an MMP-2 degradable crosslinker, an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer, optionally wherein the annealing monomer is a MethMal annealing macromer.

4. The composition of claim 3, wherein the MMP-2 degradable crosslinker comprises, consists essentially of, or consists of an amino acid sequence comprising, consisting essentially of, or consisting of Ac-GCGPQGIAGQDGCG-NH₂ (SEQ ID NO: 1).

5. The composition of claim 1, wherein the MAP scaffold has a porosity of about 10 μm to about 200 μm.

6. The composition of claim 1, wherein the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof.

7. The composition of claim 1, wherein the one or more cells that are capable of releasing insulin in response to an elevated glucose concentration are human cells or are derived from human cells.

8. A composition comprising a single cell suspension comprising, consisting essentially of, or consisting one or more cells that are capable of releasing insulin in response to an elevated glucose concentration encapsulated by microporous annealed particles (MAPs).

9. The composition of claim 8, wherein the MAPs comprise a polymer backbone comprising, consisting essentially of, or consisting of poly(ethyleneglycol) (PEG), hyaluronic acid, polyacrylamide, polymethacrylate, alginate, collagen, or any combination thereof.

10. The composition of claim 8, wherein the MAPs comprise one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer, optionally wherein the annealing monomer is a MethMal annealing macromer.

11. The composition of claim 8, wherein the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof.

12. The composition of claim 10, wherein the MMP-2 degradable crosslinker comprises, consists essentially of, or consists of an amino acid sequence comprising, consisting essentially of, or consisting of Ac-GCGPQGIAGQDGCG-NH₂ (SEQ ID NO: 1).

13. The composition of claim 8, wherein the one or more cells that are capable of releasing insulin in response to an elevated glucose concentration are human cells or are derived from human cells.

14. A method for treating diabetes, the method comprising administering to a subject with diabetes one or more of the compositions of claim 1 via a route and in an amount effective for treating the diabetes in the subject.

15. The method of claim 14, wherein the diabetes is Type 1 diabetes.

16. The method of claim 14, wherein the diabetes is Type 2 diabetes.

17. The method of claim 14, wherein the administering comprises injecting the composition into a kidney capsule, subcutaneously, intraperitoneally, into adipose tissue, intramuscularly, intrahepatically, and/or intrapancreatically into the subject.

18. The method of claim 14, wherein the composition comprises one or more cells that are capable of releasing insulin in response to an elevated glucose concentration that are human cells or are derived from human cells.

19. Use of a composition comprising a microporous annealed particle (MAP) scaffold and a single cell suspension present therein, wherein the single cell suspension comprises one or more cells that are capable of releasing insulin in response to an elevated glucose concentration, for treating diabetes, wherein the composition is formulated for administration to a subject in need thereof via a route and in an amount effective for treating the diabetes in the subject.

20. The use of claim 19, wherein the MAP scaffold comprises a polymer backbone comprising, consisting essentially of, or consisting of poly(ethyleneglycol) (PEG), hyaluronic acid, polyacrylamide, polymethacrylate, alginate, collagen, or any combination thereof.

21. The use of claim 19, wherein the MAP scaffold comprises one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer, optionally wherein the annealing monomer is a MethMal annealing macromer.

22. The use of claim 19, wherein the MAP scaffold has a porosity of about 10 μm to about 200 μm.

23. The use of claim 21, wherein the MMP-2 degradable crosslinker comprises, consists essentially of, or consists of an amino acid sequence comprising, consisting essentially of, or consisting of Ac-GCGPQGIAGQDGCG-NH₂ (SEQ ID NO: 1).

24. The use of claim 19, wherein the composition is administered administering by injecting the composition into a kidney capsule, subcutaneously, intraperitoneally, into adipose tissue, intramuscularly, intrahepatically, and/or intrapancreatically into the subject.

25. The use of claim 19, wherein the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof.

26. The use of claim 19, wherein the one or more cells that are capable of releasing insulin in response to an elevated glucose concentration are human cells or are derived from human cells.

27. The use of claim 19, wherein the diabetes is Type 1 diabetes.

28. The use of claim 19, wherein the diabetes is Type 2 diabetes.

29. A composition for use in treating diabetes in a subject in need thereof, composition comprising a single cell suspension comprising, consisting essentially of, or consisting of one or more cells that are capable of releasing insulin in response to an elevated glucose concentration encapsulated by microporous annealed particles (MAPs).

30. The composition for use of claim 29, wherein the MAPs comprise a polymer backbone comprising, consisting essentially of, or consisting of poly(ethyleneglycol) (PEG), hyaluronic acid, polyacrylamide, polymethacrylate, alginate, collagen, or any combination thereof.

31. The composition for use of claim 29, wherein the MAPs comprise one or more of a PEG-Maleimide, optionally wherein the PEG-Maleimide is a 10 kiloDalton (kDa) PEG-Maleimide; an MMP-2 degradable crosslinker; an RGD peptide, optionally wherein the RGD peptide comprises, consists essentially of, or consists of an amino acid sequence comprising, consisting essentially of, or consisting of Ac-RGDSPGGC-NH₂ (SEQ ID NO: 2); and an annealing monomer, optionally wherein the annealing monomer is a MethMal annealing macromer.

32. The composition for use of claim 31, wherein the MMP-2 degradable crosslinker comprises, consists essentially of, or consists of an amino acid sequence comprising, consisting essentially of, or consisting of Ac-GCGPQGIAGQDGCG-NH₂ (SEQ ID NO: 1).

33. The composition for use of claim 29, wherein the one or more cells that are capable of releasing insulin in response to an elevated glucose concentration are selected from the group consisting of endocrine precursor cell-derived cells and/or progeny thereof, pancreatic islet cells, and pancreatic islet-like cells derived from pluripotent stem cells (PSCs), optionally induced pluripotent stem (iPS) cells, and combinations thereof.

34. The composition for use of claim 29, wherein the diabetes is Type 1 diabetes.

35. The composition for use of claim 29, wherein the diabetes is Type 2 diabetes.


SEQUENCE LISTING (2 sequences)

SEQ ID NO: 1 - protein, 14 aa, synthetic construct
GCGPQGIAGQDGCG

SEQ ID NO: 2 - protein, 8 aa, synthetic construct
RGDSPGGC